(19) United States
(12) Patent Application Publication
(10) Pub. No.: US 2007/0218185 A1
(43) Pub. Date: Sep. 20, 2007

(54) Edible Oil Containing Statins

(76) Inventors: Christiaan Michael Beindorff, Vlaardingen (NL); Willem Maarten Meijer, Vlaardingen (NL); Henricus Otto Franciscus Molhuizen, Vlaardingen (NL)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(21) Appl. No.: 11/587,726

(22) PCT Filed: Mar. 23, 2005
(86) PCT No.: PCT/EP05/03246

§ 371 Date: Oct. 27, 2006

(30) Foreign Application Priority Data
Apr. 28, 2004 (EP) 04076293.2

(51) Int. Cl. A23D 9/007; A23C 11/02; A23D 9/02; C12P 17/06; A23L 1/16; A23C 9/152

(52) U.S. Cl. 426/601

(57) ABSTRACT

Edible oil containing statin comprising at least 90% of di- and/or triglycerides, preferably containing at least 4 mg/g statin.

### TECHNICAL FIELD

The present invention relates to an edible oil comprising statin comprising at least 90% of di- and/or triglycerides having a saturated fatty acid (SAFA) content of less than 25 wt %. Furthermore the present invention relates to a process for the preparation of such an oil. In addition, the present invention relates to the use of such an oil.

### BACKGROUND OF THE INVENTION

Cardiovascular disease is a leading cause of morbidity and mortality, particularly in the United States and in Western European countries and is emerging in developing countries. Several factors are mentioned in relation to the development of cardiovascular disease including hereditary predisposition to the disease, gender, lifestyle factors such as smoking and diet, age, hypertension, and hyperlipidemia, including hypercholesteremia. Several of these factors, particularly hyperlipidemia and hypercholesteremia, contribute to the development of atherosclerosis, a primary cause of vascular and heart disease.

Elevated low-density lipoprotein cholesterol (hereafter “LDL-cholesterol”) is directly related to an increased risk of coronary heart disease.

Statins are compounds that are known to have a lowering effect on levels of LDL-cholesterol in the human blood. Statins inhibit the hydroxymethylglutaryl coenzyme A (HMG-CoA) reductase, the rate-determining step in the cholesterol biosynthesis.

Scientific research has confirmed the healthy properties of statins especially with respect to LDL blood-cholesterol and triglyceride levels lowering activities, both in animals and in humans (Li et al., Nutrition Research 18, 71-81 (1998); Heber et al., Am. J. Clin. Nutr. 69, 231-236 (1999)).

The presence of statins in food consumed by humans is associated with a lower level of LDL-cholesterol and lower risk of coronary heart disease.

For the preparation of food containing statins, it is advantageous to have a statin source that has a high statin content and is widely applicable in food products.

WO02/64809 describes a process for the preparation of statins by fermentation and food products comprising one or more statins. It describes the extraction of statins from fermented soya beans with organic solvents (ethanol and acetonitril) and the use of the extract for the preparation of magarine and spreads. The drawback of this process is that the yield of the statins is rather low (0.0545 g statin/kg (ethanol extract) and 0.0978 g statin /kg (acetonitril extract)). Furthermore, when these extracts are used to prepare food products the extraction fluid has to be removed, which involves an extra processing step. In addition, even after vigorous evaporation and drying there may be some organic extraction fluid residue left in the extract, which is undesirable when the extract is going to be used in food products. Moreover, when considering environmental issues the use of organic solvents should be kept to a minimum.

### SUMMARY OF INVENTION

An object of the invention is to provide an edible oil containing statins. In addition, an object of the invention is to provide an oil with a high unsaturated fatty acid content and a low saturated fatty acid content. Another object of the invention is to use the edible oil containing statins for the preparation a food product. A further object of the invention is to provide a simple process containing as few steps as possible for the preparation of an edible oil containing statins.

We have now surprisingly found that extracting an oil-containing substrate which is fermented with a statins producing micro-organism with a supercritical fluid provides an edible oil containing statins.

### DETAILED DESCRIPTION OF THE INVENTION

The preparation of purified statins is known from the pharmaceutical industry. The preparation and purification of the statins used in pharmaceutical preparations involves many process-steps, in which ingredients are used that are not commonly used in the food industry. The many process steps are costly compared to processes having less process steps. For these reasons the statins prepared for pharmaceutical use are not used in the foods industry. Synthetic or half-synthetic statins, e.g. as used in the pharmaceutical industry, are also less desirable in food products.

In WO02/063976, a functional food product comprising soy protein and statin is described, that reduces the low-density lipoprotein cholesterol level in human beings. The food products may be prepared by fermenting soy with one or more filamentous fungi. These products contain both soy protein and statin, thereby limiting statin containing products to products also containing soy protein. In addition the amount of statin in the products is such that a large amount of these products has to be used to obtain a LDL-cholesterol lowering effect. For a more general application in the food industry, it is advantageous to have a product that has a large concentration of statin and contains as few other compounds as possible.

The following definitions will be used:

Statins are defined as substances having the structural formula, presented in FIG. 1. In this structural formula, R1 and R2 can be any group. Preferred statins are those which are given in FIG. 1.

The amounts given will be expressed, in wt. % or weight parts per million (ppm), mg/kg or mg/g, relative to the total weight of the food product, unless otherwise indicated.

The amounts of statins given herein are the sum of the amounts of individual statins, as e.g. determined by chromatography, unless otherwise indicated.

An object of the invention relates to a process for the preparation of an edible oil containing statin. Statin containing oil may be obtained by extraction with super critical fluid of a substrate which is fermented with a statin producing fungus.

A supercritical fluid is formed when a gas is compressed at a temperature too high to form a liquid. Above a certain temperature, called the critical temperature, the thermal kinetic energy of the gas molecules is always higher than the attraction energy between the molecules. Above this temperature, no pressure is high enough to condense the vapour into a liquid. A certain minimum pressure, called the critical pressure, is necessary to form a supercritical fluid. Below this critical pressure, the component is behaving as a gas. For a pure component, there is no difference any more between gas and liquid at conditions above its critical temperature and pressure. This is reflected in a combination of a low, gas-like viscosity and a high, liquid-like density.

Many gases may be used in super critical fluid extractions, like the noble gases, other gases like nitrogen, hydrogen, and oxygen, alkenes like ethene and propene, alkanes like methane, ethane, propane, and butane, alkynes and alkylhalides like tetrafluoro methane. Very suitable gases are those approved by the FDA as safe human food ingredients, like carbondioxide, nitrogen, helium, propane, n-butane, iso-butane, and nitrous oxide (N₂O). For application in food processing, carbon dioxide is especially suitable for use in supercritical extraction. Carbon dioxide has a relatively low supercritical temperature and pressure and it is cheap, non-toxic and easily removed.

Optionally, inorganic and/or organic compounds may be added to the supercritical fluid. These modifiers or co-solvents employed in the process should be compatible with the supercritical fluid selected and also be capable of at least partially dissolving the compounds being extracted. Ethanol, acetone, water, diluted acids or bases and ethanol/water (50/50 v/v) mixtures are suitable co-solvents.

In a preferred embodiment no co-solvent is used in the extraction process. Preferably, the supercritical fluid is free of co-solvents. The supercritical fluid is food grade and its purity is preferably 99% or more.

The substrate may be contacted with the super critical fluid at temperatures ranging from 20 to 95° C., preferably 30 to 60° C. The pressure should be sufficient to maintain the supercritical fluid, and may be increased from 75 to about 550 bar, preferably between 150 and 400 bar.

The choice of the reaction parameters will vary depending on the super critical fluid and modifier used. The skilled person will be able to determine which conditions to use based upon the known properties of the substrate, the compound to be extracted as well as the gas specifications, including supercritical temperature and pressure.

The substrate may vary depending on whether it may be fermented with a statin producing fungus.

It has been shown that statins can be produced by a variety of filamentous fungi, including Monascus, Aspergillus, Penicillium, Pleurotus, Pythium, Hypomyces, Paelicilomyces, Eupenicillium, and Doratomyces.

As a food product, rice fermented with a red Monascus fungus (red rice) has been known and used for hundreds of years in China. Red rice was used and still is used in wine making, as a food-colouring agent and as drug in traditional Chinese medicine. We have found that most red rice available on the market contains no statins or statins in very low amounts. The Food and Drug Administration has concluded that red yeast rice available in the market does not contain significant amounts of lovastatin (FDA, Docket No. 97-0441, Final Decision).

WO 99/23996 describes a composition for treating elevated serum cholesterol and/or triglycerides comprising a red rice product containing at least 0.05% lovastatin by weight.

Red rice powder capsules are sold as dietary supplements under the name of Cholestin by the firm Pharmanex. Pharmanex also sells a Cholestin bar containing red yeast rice (Monascus purperus went).

Red rice has an intensive red colour. Whereas the intensive red colour of red rice is an advantage when it is used as colouring agent, it is a disadvantage when it is used in food products. Due to the intense red colour of red-rice products, the foods prepared from red rice are coloured, depending on the amount of red-rice product added to the food product yellow, orange or red. The higher the amount of red rice added to the food, the more intense is the red colour of the food product. In the known food products a relatively large amount of red rice has to be added in order to add enough statins. This results in a red colour of the products that cannot be avoided.

In some food products the red rice colouring is undesirable. In particular in the western world, consumers are reluctant to use products of which the colour has changed from that they are used to. For example spreads, including margarine, butter, low fat spreads or salad oils are considered unacceptable by customers, when the colour of such a product is orange or red. However, at the same time these type of products have been found by us to be excellent vehicles of the daily intake of amounts of statins sufficient to obtain a blood LDL-cholesterol lowering effect.

Preferably the fungus is chosen from the group consisting of Monascus fungi and more preferably from the group consisting of Monascus ruber fungi.

Most preferably the fungus is Monascus ruber F125 M1-4, which gives no red coloring when grown on soy material.

The fermentation may be carried out in a manner, which can be determined by the skilled person on the basis of the methods described in WO02/064809 and WO02/063976.

The fermentation temperature may be important. The temperature is preferably in the range of 10 to 37° C., more preferably 20 to 30° C.

Preferably during fermentation the medium is aerated, e.g. by stirring, shaking etc. Aeration may be carried out by blowing air through the fermentation medium. Preferably the air is wholly or partly saturated with water vapour in case solid state fermentation is used. This avoids drying out of the fermentation medium.

The levels of statins will depend on the fermentation time. The fermentation time is therefore dependent on the desired amount of statins. Preferred fermentation time is 1-50 days, more preferably 15-40 days, most preferably 20-30 days (See WO02/064809 and WO02/063976).

In addition, the substrates preferably contain oil that can be extracted together with the statin produced by the fungus. Suitable substrates that may be used are soybeans, nuts like hazelnuts, wallnuts, and peanuts, olives, sunflower seeds, rapeseeds, rice, kidneybeans, mungbeans, lupine seeds, maize, or oat.

The substrate preferably contains oil with a high poly-unsaturated fatty acid (PUFA) content and a low saturated fatty acid (SAFA) content.

Especially suitable substrate are soybeans. Soybean oil has little flavor, which is an advantage because it does not interfere with the taste of the food. Soybean oil is the most commonly used oil in food manufacturing. Soybean oil is adaptable to nearly every fat or oil application in the food industry. It works well with other ingredients, including other fats and oils, making it very suitable for use in fat-based foods e.g. spreads, salad dressings, sauces and baked goods.

The process of the present invention provides for an edible oil containing statin. An edible oil is defined as an oil or fat which is suitable for human consumption. The expression oil as used in the present application includes both solid fat and liquid oil. The edible oil comprises of more than 90 wt % of di- and/or triglycerides. The oil is ready to use for the preparation of food products and in the case with liquid oils as table oil. The oil according to the invention may be any edible oil depending on the substrate used, e.g. soybean oil, olive oil, sunflower oil, or rapeseed oil.

Preferably the edible oil comprises at least 1 mg/g statin, and more preferably at least 4 mg/g statin.

In addition to the oil and statins other compounds that are beneficial for hearth health, such as for instance polyphenols, polyunsaturated fatty acids, phytosterols, peptides, tocopherols, saponins, dietary fibers and vitamins may be extracted together with the oil and statins from the fermented substrate.

Polyphenols herein are polyphenols having plant origin. These include flavenoids, which include isoflavones. The polyphenols include isoflavones, stilbenes, lignans, coumestans and resorcyclic acid lactones. Examples of isoflavones are genistein, daidzein, equol, glycitein, biochanin A, coumestrol, maitaresinol, formononetin, O-desmethylengolesin, enterolactone and enterodiol. Preferred isoflavones according to the invention are genistein and daidzein and glycitein, which are present in soybeans.

Saponins are herein derived as β-D-glucopyranosiduronic acid derivates. Examples of saponins are Soya sapogenol A,B,C,D and E, Soyasaponin I, II and III, as described in Lebensmittel Lexikon, B.Behr's Verlag GmbH & Co. Hamburg, Bd.2, L-Z -3, 1993, pages 550-552.

Polyunsaturated fatty acid esters are defined as fatty acid esters having more than one unsaturated group in the fatty acid chain. Examples of polyunsaturated fatty acid esters are linoleic acid esters, linolenic acid esters, arachidonic acid esters.

Dietary fibers are herein a collective term for a variety of plant substances, that are resistant to digestion by the human gastrointestinal enzymes. Depending on their solubility, dietary fibers can be classified into insoluble (cellulose, some hemicelluloses, lignins), and soluble (remainder of the hemicelluloses, gums, mucilages. Soybean colyledon fibers comprise both soluble and insoluble dietary fibers.

Phytosterols are herein defined as sterol compounds produced by plants, which are structurally very similar to cholesterol except that they contain some substitutions at the C24 position on the sterol side chain. The phytosterols include 4-desmethylsterols, 4-monomethylsterols, 4,4′-dimethylsterols and mixtures thereof. Examples of such phytosterols are β-sitosterol, campesterol, stigmasterol. The term phytosterols herein also includes phytostanols, the saturated equivalents of phytosterols.

Tocopherols are members of the vitamin E family. The term vitamin E includes eight naturally occurring isomers with widely varying degrees of biological activity. Four are in the form of tocopherols (a, b, g, d); The remaining four are in the form of tocotrienols (a, b, g, d).

The role of Vitamin E is unique and indispensable. Its structure allows it to position itself strategically and protect the cell and other membranes. It also protects LDL and other lipids from oxidation.

Gamma-tocopherol, is the effective form that fights nitrogen free radicals. These radicals are major culprits in arthritis, multiple sclerosis (MS) and diseases of the brain (such as Alzheimer's). A metabolic product of gamma-tocopherol appears to help regulate the amount of fluid and electrolytes that pass through the kidney and end up in urine. Thus, it could play a major role in blood pressure control, congestive heart failure, and cirrhosis of the liver.

The National Academy of Science's Recommended Daily Intake (RDI) for vitamin E is 15 milligrams. International units are used as a measure of alpha tocopherol. The IU is based on alpha-tocopherol acetate. 1 mg alpha-tocopherol acetate corresponds to 1.0 IU alpha-tocopherol acetate, and 1 mg alpha-tocopherol=1.49 IU alpha tocopherol.

Furthermore alpha-tocopherol equivalent (alpha-TE) is also used for a measure of vitamin E. The alpha-tocopherol equivalent takes into account all 8 members of the vitamin E family in foods.
alpha-tocopherol equivalent=(mg alpha)+(0.4 mg beta)+(0.01 mg gamma)+(0.1 mg delta).

The recommended daily intake (RDI) of tocopherol is 10 a-tocopherol equivalents/day

In another embodiment the present invention relates to an edible oil comprising statin comprising at least 90% of di- and/or triglycerides and 50 to 1000 alpha tocopherol equivalents per kg, preferably 100 to 750 alpha tocopherol equivalents, even more preferably 250 to 750 alpha tocopherol.

A suitable amount of total tocopherol is 500 to 10000 mg/kg. total tocopherol is the sum of all the tocopherol present. Preferred amounts are 750 to 5000 mg/kg, more preferred 1000 to 2500 mg/kg.

Suitably the edible oil of the present invention has a saturated fatty acid (SAFA) content of less than 25 wt %, preferably less than 20 wt %, more preferably less than 15 wt %. Furthermore the edible oil according to the present invention has an unsaturated fatty acid (UFA) content of at least 75 wt %, at least 80 wt % and even more preferably, at least 85 wt %. It is desirable for the edible oil of the present invention to have a polyunsaturated fatty acid (PUFA) content of more than 5 wt %, preferably more than 15 wt %, more preferably more than 30 wt % and most preferably more than 50 wt %.

It is preferred that the edible oil of the present invention has less than 10 mg/kg of cholesterol.

Another object of the invention is to use the edible oil for the preparation a food product. Several food products may be prepared according to the invention, for example, spreads, magarines, soups, pasta, noodles, ice-cream, sauces, dressing, mayonnaise, snacks, cereals, beverages, bread, biscuits, other bakery products, sweets, bars, chocolate, chewing gum, dairy products, dietetic products e.g. slimming products or meal replacers etc. In particular pasta, soya-milk or cow-milk may be prepared according to the invention.

The food product according to the invention preferably comprises statins in an amount sufficient to obtain a blood LDL-cholesterol lowering effect if the food product is used according to the common needs of the consumer.

The preferred intake of statin per day is herein 5-40 mg/day, more preferably 5-20 mg/day, even more preferably 8-15 mg/day. Furthermore, the intake of statin per day is preferably 1-5 mg/day, more preferably 1-2.5 mg/day.

The skilled person will be able to adjust the percentage of statins in the food product to obtain the above effect. The percentages will depend on the type of food product, since the food products are used in different serving sizes. Moreover the pattern in a food product is consumed (servings per day and distribution over days) is dependent on the food product.

Preferably the food product according to the invention comprises statin and non-glycosylated isoflavone. In soy beans and soy materials derived from soy, isoflavones are present substantially in the glycosylated form. Typically about 5 wt. % of the isoflavones is present in the non-glycosylated form. The most important glycosylated isoflavones are genistin, daidzin and glycetin. The non-glycosylated forms are respectively genistein, daidzein and glycetein. Genistein, daidzein and glycetein have been reported to have advantageous health effects, including estrogenic and antioxidant properties.

We have found that due to the fermentation according to the invention the glycosylated isoflavones are converted into the corresponding non-glycosylated isoflavones, which are more benificial. For instance, the amount of genistein and daidzein is increased in the fermented soy compared to the non-fermented soy. Surprisingly this advantageous conversion occurs simultaneously with the production of statin.

When colours are classified, they can be broken down into the three primary elements. One is the Hue (colour) the other is Value (brightness) and the third is Chroma (Saturation like vivid colours or dull colours).

To enable anyone to tell anyone else exactly what colour they are talking about a common numerical code is used. This numerical code used is L*a*b*. When a colour is expressed in this system, Value becomes L*, while Hue and Chroma are expressed as a* and b* respectively. The L*a*b* may be measured with a UV 1601 spectofotometer of Shimatzu:

Preferably the food product has a Hue a* value of less than 20, preferably less than 10, most preferably less than 0.

### DESCRIPTION OF THE DRAWING

FIG. 1 shows a schematic representation of the structure of different statins.

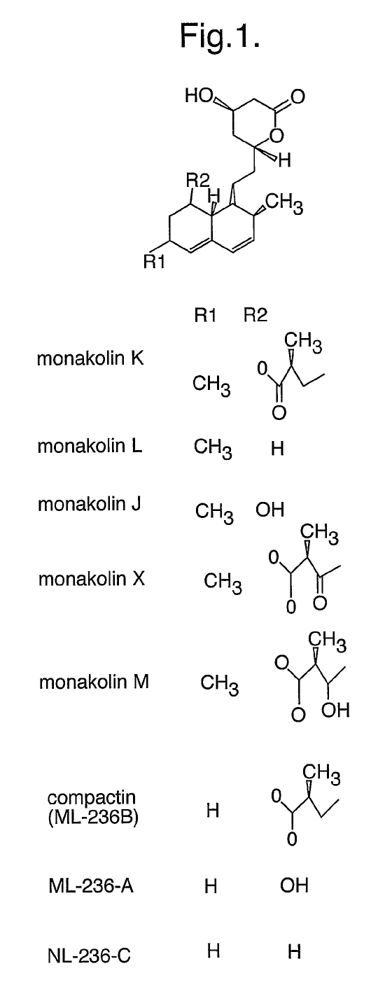

FIG. 2: shows a schematic representation of the experimental set-up for supercritical carbon dioxide extraction.

### EXAMPLES

General:

Supercritical Extraction

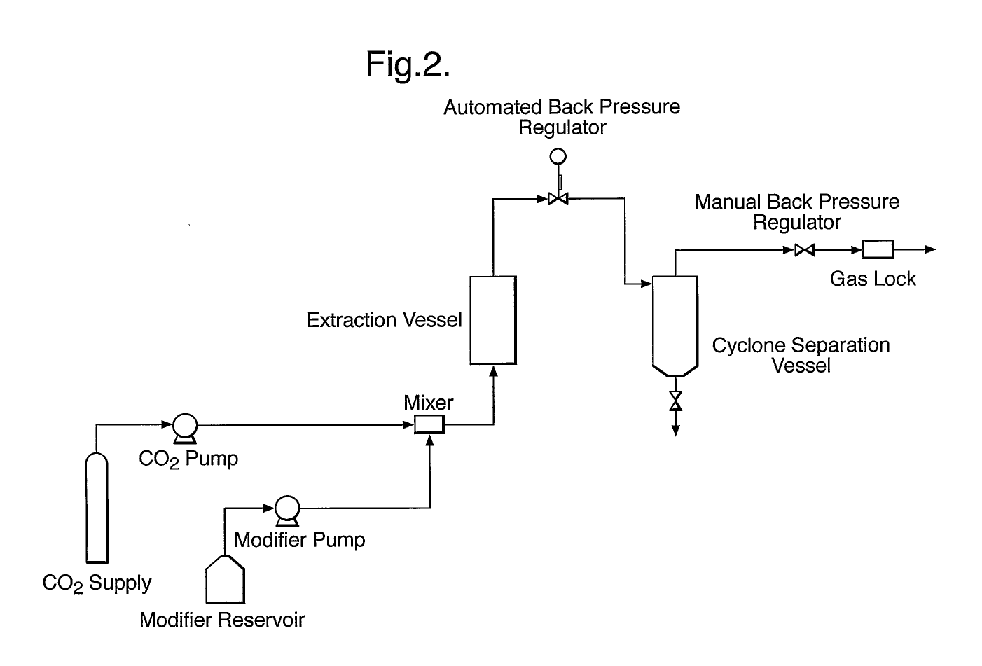

For the supercritical extraction of natural solid matrices, equipment of Thar Designs was used. The experimental set-up is schematically depicted in FIG. 2.

The CO₂ pump is capable of compressing liquid carbon dioxide to a pressure up to 600 bars at a constant flow-rate. In a static mixer, a polarity modifier may be mixed with the liquid carbon dioxide. The maximum pressure that can be applied in the presence of a modifier is about 400 bar. In a pre-heater (not depicted) the carbon dioxide (+modifier) was heated to reach supercritical conditions before entering the extraction vessel.

In the extraction vessel, which was heated with a double wall heating mantle, the supercritical carbon dioxide was passed over the solid matrix for extraction.

Downstream of the extraction vessel, the supercritical carbon dioxide was expanded over an automated backpressure regulator. The backpressure regulator was coupled to a feedback control unit to control the pressure in the system. The carbon dioxide was separated from the extracted material (liquid/solid) in a cyclone separation system. The carbon dioxide left the cyclone at the top, while the extracted material remained in the cyclone. The liquids extracted from the solid matrix were recovered during the experiment by opening the valve at the bottom of the cyclone.

The carbon dioxide gas was further expanded over a further backpressure regulator, which was operated manually. A gas clock downstream of the backpressure regulator registers how much gas has been put through the system, before the carbon dioxide leaves the system at ambient pressure.

The process equipment is designed to operate at the following conditions:

TABLE 1
Process operation conditions supercritical extraction set-up

| Parameter | Range |
|---|---|
| Flow rate of liquid | 5-150 g/min |
| carbondioxide | |
| Pressure | up to 600 bar (with modifier: |
| | up to 400 bar) |
| Temperature | 20-100° C. |
| Extractor volume | 25-500 mL |
| Cyclone volume | 25 or 200 mL |
| Matrix to be | solid or liquid |
| extracted | |
| Extract | liquid or solid at ambient |
| | temperature and pressure |
| Modifier addition | flow rate 0-10 mL/min |

Determination of Lovastatins Using HPLC

Samples were prepared by adding 25 ml of an extraction mixture, containing acetonitrile, water and phosphoric acid (1:1:0.05, v/v/v) to approximately 5 g of soybeans. Statin in oil samples (40-100% oil) and liquid samples (like soymilk) were extracted by the addition of 100% acetonitrile in a 1:1 ratio (v/v). Samples were incubated for 1 hour at room temperature and then homogenised using an Ulta-Turrax. After homogenisation the samples were incubated overnight at room temperature on a roller bench. The samples were centrifuged at 11.000 rpm for 10 minutes and the supernatants collected for HPLC analysis. The amount of lovastatin in the samples was determined by HPLC separation according to the method of Morovjan et al. J. chromatogr. A 763 (1997) 165-172.

The system consists of the Shimadzu SCL-10A system controller, CTO-10AS column oven, LC-10AT vp pump system, RID-10A refraction index detector, SPD-M10A diode array detector and SIL-10AD autoinjector. For the chromatographic determination of lovastatin a Waters NovaPak C18 (150×3.9 mm I.D., 4 μm) column was used operating at 25° C. The eluent was acetonitril-0.1% phosphoric acid (50:50, v/v) solution flowing at 1.5 ml/min. Runs were performed for 15 min. The detection was performed using a diode array detector from 190 nm up to 800 nm. The sum of the area of all peaks in the spectrum belonging to lovastatin is measured. Comparison to a standard (Mevinolin, Sigma) allows the calculation of a lovastatin content (expressed in mg/g analysed product).

### Example 1-8

### Preparation of Fermented Substrates

Inoculum Preparation

Monascus ruber F125 M1-4 was plated on VMA-agar plates and incubated at 30° C. for 3 days.

With a sterile scalpel, small squares were cut in the VMA-agar for the preparation of inoculates. With a sterile spatula, the blocks of agar were transferred to the liquid media. Malt water was used for pre-cultivation. Sterile flasks of 500 ml were filled with 300 ml medium. The flasks were incubated in an Innova 400 shaker at 25° C. for 2 days.

Strains F125 and F125 M1-4 are deposited at the Centraal Bureau voor Schimmelculturen (CBS) as no. CBS 109070 on 14.11.2000 and no. CBS 109269 on 23.01.2001.

These deposits were made under the provisions of the Budapest Treaty on the International Recognition of the Deposit of Microorganisms for the Purpose of Patent Procedure and the Regulations thereunder (Budapest Treaty).

Fermentation Process

Substrates like soybeans, kidney bean, mung bean, lupine seeds, walnut, maize, oat and peanuts were soaked in tap water (50° C.) for 60 minutes. After soaking the substrates were rinsed with cold tap water. Subsequently the substrates were air-dried at ambient temperature for 180 minutes. The soaked and dried substrates were transferred to a shake-flask, approximately 50 g per flask. The shake flasks were sterilised by autoclaving (10 min 120° C.), inoculated with 1 ml of a fully-grown Monascus culture and incubated for an appropriate time (2-6 weeks) at 25° C. Lovastatin production is monitored and when a sufficient level has been obtained, the flasks are pasteurised by placing the flasks in an incubator at 80° C. overnight after which the end product can be harvested. Table 2 gives an overview of the statin content of the end product after 3 weeks of fermentation.

TABLE 2
Amount of statin in end product after 3 weeks of fermentation

| | | Statin |
|---|---|---|
| Example | Substrate | g/kg |
| 1 | Soy beans | 1.2 |
| 2 | Kidney beans | 1.2 |
| 3 | Mung beans | 1.2 |
| 4 | Lupine seeds | 1.5 |
| 5 | peanut | 0.4 |
| 6 | Walnut | 0.8 |
| 7 | Maize | 0.3 |
| 8 | Oat | 1.4 |

### Example 9

### Supercritical Extraction of Fermented Soybeans

The fermented soybeans were ground prior to extraction in water-cooled universal mill (type M20, IKA, Germany) until a fine powder was obtained.

The amount of sample used for extraction was 100 gram of ground fermented soybeans. The fermented and ground soybeans were put in the extraction vessel (500 ml) and the remaining volume was filled with small glass beads (2 mm diameter). The total solvent flow rate was 20 g·min⁻¹. In the case of addition of modifier, 18 g·min⁻¹ carbon dioxide was mixed with 2 g·min⁻¹ modifier (ethanol or ethanol/water mixture). The flow rates were adjusted using the control software supplied with the Thar extraction equipment. At time intervals of 30 minutes, the cyclone separation vessel was opened and the extracted oil was collected. The total extraction time was 2 hours. The process is suitable for industry-scale extraction.

In the case when ethanol was used as modifier, the ethanol was removed by evaporation under vacuum with a rotary evaporator until constant weight. When ethanol/water 50/50 (v/v) mixture was used as polarity modifier, the oil phase was separated from the ethanol/water phase by centrifugation at 3200 g for ten minutes in a centrifuge equipped with a swing-out rotor. Aliquots of the collected oil fractions, water phases and also of the residue material from the extractor were analysed for lovastatin content, see Table 3.

TABLE 3
Results from super critical carbon dioxide extraction of
Monascus fermented soybeans.

| | | | Lovastatin | Total | |
|---|---|---|---|---|---|
| | | Mass | content | Lovastatin | |
| | Time | extracted | extracted | extracted | Recovery |
| | interval | oil | oil | oil | of statin |
| Description | [minutes] | [g] | [mg/g] | [mg] | [%] |
| Start | | 100.0 | 1.2 | 121 | 100 |
| material | | | | | |
| Fraction 1 | 0-30 | 13.4 | 4.5 | 60 | 50 |
| Fraction 2 | 31-60 | 3.3 | 1.6 | 5 | 4 |
| Fraction 3 | 61-90 | 1.8 | 1.6 | 3 | 2 |
| Fraction 4 | 91-120 | 0.7 | 2.0 | 1 | 1 |

The CO₂-extracted fermented soy oil was analysed for phytosterol and tocopherol content, the results are shown in Table 4.

TABLE 4
tocopherol and phytosterol content
of CO₂-extracted fermented soy oil

| material | [mg/kg] |
|---|---|
| a-Tocopherol | 100 |
| b-Tocopherol | 18 |
| g-Tocopherol | 905 |
| d-Tocopherol | 481 |
| Cholesterol | <10 |
| Cholestanol | 22 |
| 24-Methyl cholesterol | 132 |
| Campesterol | 958 |
| Campestanol | 44 |
| Stigmasterol | 822 |
| Sitosterol | 1733 |
| Sitostanol | 262 |
| D5-avenasterol | 85 |

### Comparative Example A, B, C

### Soxhlet Extraction with Different Solvents

For use in food applications, the extraction of statins from fermented soybeans can be performed with a number of organic solvents: hexane, acetone, ethyl acetate and ethanol or mixtures of these.

For testing the extraction with different organic solvents classical Soxhlet extraction was performed. Approximately 135 g of ground fermented soybeans was put in an extraction thimble. Approximately 500 ml of organic solvent was added and the extraction was performed for 3 hours. After the extraction the organic solvent was evaporated under reduced pressure with a rotatory evaporator until constant weigh. An aliquot was taken for the determination of the statin concentration using reversed phase chromatography (HPLC, Shimadzu). Results are shown in Table 5.

TABLE 5
Result of soxhlet extraction of Monascus fermented soybeans.

| | | | Total amount of | | Total amount of | |
|---|---|---|---|---|---|---|
| | | Lovastatin content | lovastatin in | Lovastatin content | lovastatin in | Recovery |
| | Extraction | in starting product | starting product | of extracted oil | extracted oil | Lovastatin |
| Example | solvent | (mg/g) | (mg) | (mg/g) | (mg) | (%) |
| A | Hexane | 0.69 | 93 | 1.13 | 22.6 | 24 |
| B | Ethanol | 0.79 | 106 | 1.77 | 19.5 | 18 |
| C | Acetone | 0.73 | 98 | 0 | 0 | 0 |

As can be seen from table 3 and 5, the recovery of statin is much higher when the fermented soybeans are extracted with super critical carbon dioxide than with organic solvents.

### Example 10

### Preparation of a Food Product: Statin Containing Cow-Milk

Statin containing low fat (1.8%) cow-milk was prepared through addition of 2.7 g soy oil containing lovastatin (1 mg/g) and 15.3 g sunflower oil to one litre of sterile skimmed milk. The mixture was homogenised and pasteurised prior to packing. The level of lovastatin in the end product is 2.7 mg/L. Daily consumption of 200 ml would provide an estimated Blood Cholesterol lowering (BCL) of 3%.

Cow-milk with a higher statin level was prepared through addition of 12.5 g of statin containing soy oil and 5.5 g sunflower oil to one litre of sterile skimmed milk. The level of lavastatin in the end product is 12.5 mg/L. Daily consumption of 200 ml would provide an estimated BCL of 10-15%.

### Example 11

### Preparation of a Food Product: Statin Containing Soy-Milk

Soy-milk containing statins was prepared by addition of 2.5 g soy oil with lovastatins (1 mg/g) to one litre of soy milk prepared from commercially available soy-milk base (AdeS). Estimated percentage fat in the final product is 2.4%. The amount of Lovastatin in the product is 2.5 mg/L. Daily consumption of 200 ml would provide an estimated BCL of 3%.

### Example 12

### Preparation of a Food Product: Statin Containing Dried Cow-Milk and Dried Soy-Milk

225 g of Maltodextrin (Passelli) was added to a litre soymilk/cows milk containing lovastatin and the milk was spray dried using a labscale spray dryer (Buchi). Settings:
Inlet temperature=130° C.
Outlet temperature=90° C.
Aerator=15 (75%)
Nozzle pressure=4 bar
Flow=200 ml/h

The estimated amount of statin in the milk powder was 0.011 mg/g.

### Example 13

### Preparation of a Food Product: Statin Containing Pasta

The pasta was prepared from the ingredients in Table 6. The estimated amount of Lovastatin in the pasta is 0.013 mg/g. The consumption of 80 g would result in an estimated BCL of 5%.

TABLE 6
Ingredients for pasta

| Ingredient | amount (g) |
|---|---|
| Flour | 2650 |
| Tapioca starch | 300 |
| Oil with statins | 45 |
| sodium- alginate | 2.2 |
| Titanium dioxide | 0.2 |
| CaCl2 | 0.2 |
| water | 517 |

1. Edible oil comprising statin comprising at least 90% of di- and/or triglycerides having a saturated fatty acid (SAFA) content of less than 25 wt %.

2. Edible oil according to claim 1 having a unsaturated fatty acid (UFA) content of at least 75 wt %.

3. Edible oil according to claim 1 having a polyunsaturated fatty acid (PUFA) content of more than 5 wt %.

4. Edible oil according to any of claim 1 having 50 to 500 alpha tocopherol equivalents per kg.

5. Edible oil according to claim 1 comprising at least 1 mg/g statin.

6. Edible oil according to claim 5 comprising at least 4 mg/g statin.

7. Edible oil according to any of the claim 1 having less than 10 mg/kg of cholesterol.

8. Process for the preparation of an edible oil comprising statin characterised in that the process comprises extracting a substrate which is fermented with a statin producing fungus with super critical fluid.

9. Process according to claim 8 wherein the super critical fluid is super critical CO₂.

10. Process according to claim 8 wherein the supercritical fluid is free of co-solvent.

11. Process according to claim 8 wherein the substrate are soybeans.

12. Process according to claim 8 wherein the statin producing fungus is a Monascus fungus.

13. Process according to any claim 8 comprising the steps of
fermenting a substrate with a statin producing fungus,
grounding the substrate,
extracting the substrate with super critical fluid,
and recovering the oil.

14. Use of an edible oil according to claim 1 in the preparation of a food product selected from the group consisting of pasta, soya-milk, cow-milk, and dried milk powder.
